(19) United States
(12) Patent Application Publication
(10) Pub. No.: US 2020/0221998 A1
(43) Pub. Date: Jul. 16, 2020

(54) POISON DIAGNOSTIC DEVICE, SYSTEM, AND METHOD OF USE

(71) Applicants: Omid Mehrpour, Denver, CO (US); Shiva Shabrang, Denver, CO (US)

(72) Inventors: Omid Mehrpour, Denver, CO (US); Shiva Shabrang, Denver, CO (US)

(21) Appl. No.: 16/527,627

(22) Filed: Jul. 31, 2019

Related U.S. Application Data
(60) Provisional application No. 62/790,824, filed on Jan. 10, 2019.

(51) Int. Cl. A61B 5/00; G16H 20/10; G06N 20/00; G16H 50/20

(52) U.S. Cl. CPC A61B 5/4845; G16H 20/10; G16H 15/00; G16H 50/20; G06N 20/00

(57) ABSTRACT

A poison diagnostic system includes a central processing unit in communication with a database; an interface to provide a display, the interface to communicate with the central processor; data stored within the database and relating to poison symptoms; and the interface having input criteria associated with poison symptoms; the central processing unit is to receive a data points associated with the input criteria and correlate the data points to the database to provide an output; and the output is a diagnostic sheet providing a correlation between the data points and possible poison causes.

### BACKGROUND

### 1. Field of the Invention

The present invention relates generally to diagnostic systems, and more specifically, to a poison diagnostic device, system, and method for providing one or more possible poisons that could be a cause of symptoms.

### 2. Description of Related Art

Diagnostic systems are well known in the art and are effective means to receive medical care. For example, FIG. 1 depicts a flowchart 101 of a conventional diagnostic system, wherein the patient seeks out a physician and provides the physician with their symptoms, as shown with boxes 103, 105. The physician then relies on their training and experience to diagnose if the symptoms correlate to one or more poisons, as shown with boxes 107, 109.

One of the problems commonly associated with system 101 is limited training and experience. For example, the physician may not know symptoms associated with all possible causes.

Accordingly, although great strides have been made in the area of diagnostic systems, many shortcomings remain.

### DESCRIPTION OF THE DRAWINGS

The novel features believed characteristic of the embodiments of the present application are set forth in the appended claims. However, the embodiments themselves, as well as a preferred mode of use, and further objectives and advantages thereof, will best be understood by reference to the following detailed description when read in conjunction with the accompanying drawings, wherein:

FIG. 1 is a flowchart of a common diagnostic system;

FIG. 2 is a simplified schematic of a poison diagnostic system in accordance with a preferred embodiment of the present application;

FIG. 3 is an exemplary list of input criteria received in the system of FIG. 2;

FIG. 4 is an exemplary output as provided by the system of FIG. 2;

FIG. 5 is a flowchart of a method of use of the system of FIG. 2;

FIG. 6 is a schematic of resources contemplated for use with the system of the present invention; and

FIG. 7 is a flowchart of a method of use of the resources of FIG. 6.

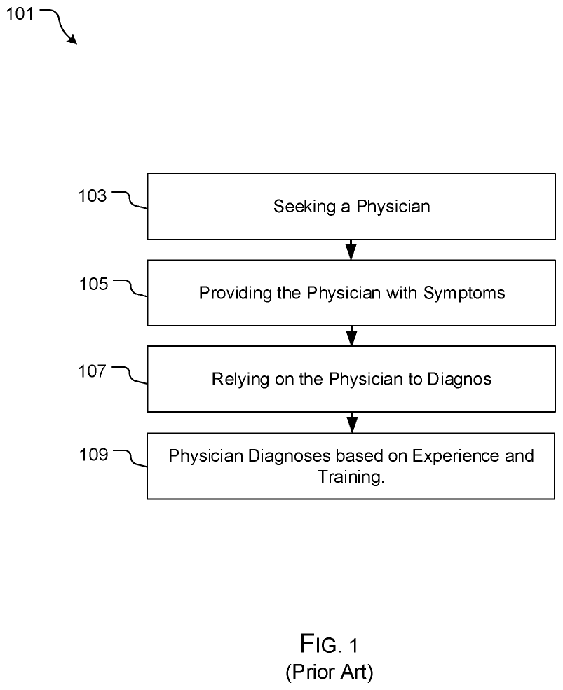

While the system and method of use of the present application is susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the present application as defined by the appended claims.

### DETAILED DESCRIPTION OF THE PREFERRED EMBODIMENT

Illustrative embodiments of the system and method of use of the present application are provided below. It will of course be appreciated that in the development of any actual embodiment, numerous implementation-specific decisions will be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The system and method of use in accordance with the present application overcomes one or more of the above-discussed problems commonly associated with conventional diagnostic systems. Specifically, the present invention provides an output based on data points received correlating to poison symptoms. These and other unique features of the system and method of use are discussed below and illustrated in the accompanying drawings.

The system and method of use will be understood, both as to its structure and operation, from the accompanying drawings, taken in conjunction with the accompanying description. Several embodiments of the system are presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless described otherwise.

The preferred embodiment herein described is not intended to be exhaustive or to limit the invention to the precise form disclosed. It is chosen and described to explain the principles of the invention and its application and practical use to enable others skilled in the art to follow its teachings.

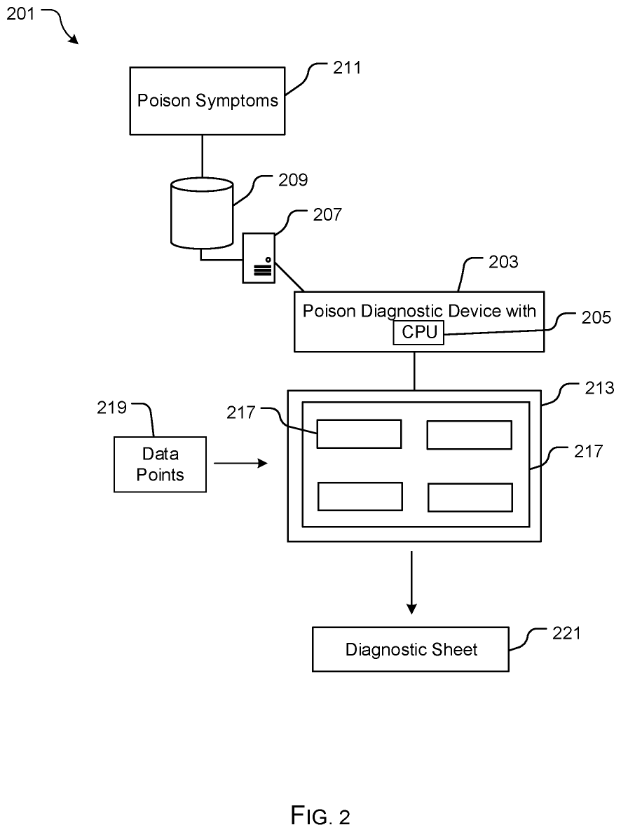

Referring now to the drawings wherein like reference characters identify corresponding or similar elements throughout the several views, FIG. 2 depicts a simplified schematic of a poison diagnostic system in accordance with a preferred embodiment of the present application. It will be appreciated that system 201 overcomes one or more of the above-listed problems commonly associated with conventional diagnostic systems.

In the contemplated embodiment, system 201 includes a diagnostic device 203 having a CPU 205 in communication with one or more of a server 207 and a database 209. It should be appreciated that the components can be built into one machine, or alternatively, can be machines configured with wireless or wired communication.

The database includes a plurality of data 211 associated with poison symptoms. In the preferred embodiment, the poison diagnostic device 203 further includes, or is in communication with, an interface 213 having a display 215 with a plurality of input criteria 217 configured to receive data points 219. It should be appreciated that the input criteria is configured to correlate to the database of poison symptoms, wherein the user (physician or other personnel) will input data points, and the CPU processes the data points to provide an output 221. In the preferred embodiment, the output 221 is a diagnostic sheet, which can either be physical or electronic, the diagnostic sheet provides the results of the processing of the data points.

It should be appreciated that one of the unique features believed characteristic of the present application is the collection of data to provide an output relating to possible poison causes.

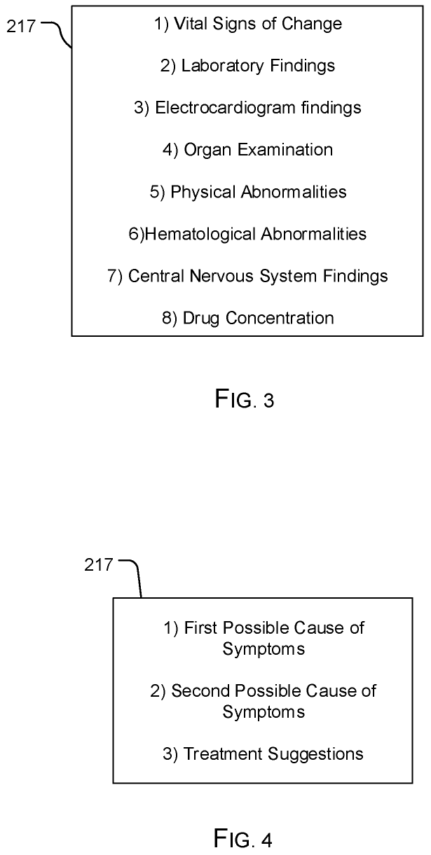

In FIG. 3, one example of a plurality of input criteria 217 is shown. It is contemplated that the physician (or other personnel) will input symptoms of a patient. Some of the input symptoms will include vital signs change, such as tachycardia, bradycardia, hyperthermia, hypothermia, hypertension, hypotension, respiratory rate changes; laboratory finding, such as blood glucose level, lactate level, serum BUN and creatinine, serum sodium and potassium; electrocardiogram finding such as (ST-t changes, PR interval, QT interval, QRS interval,etc.,); organ examination; physical abnormalities such as hearing loss, visual disturbance, tinnitus, etc.; hematologic abnormalities (thrombocytopenia, leukocytosis, leukopenia, anemia, etc.); central nervous system finding such as seizure, mental status changes, etc.); and drug concentration (if any). In addition, there are many other signs and symptoms that can be included.

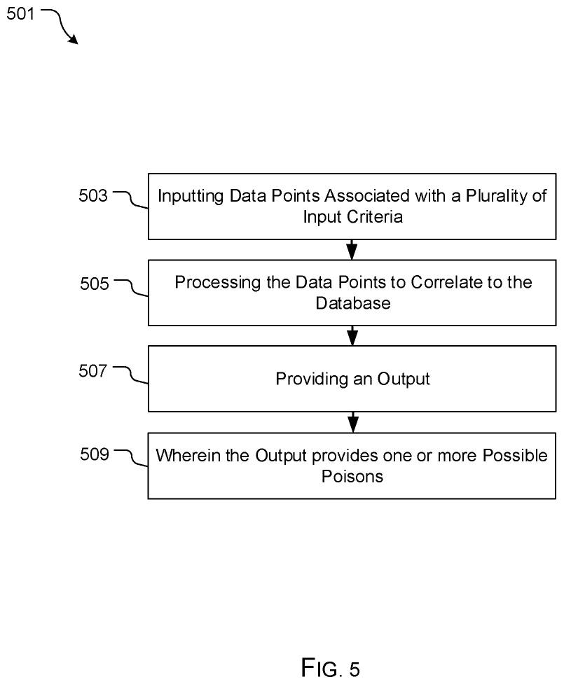

In FIG. 4, one example of an output 221 is shown. The output can include a first possible cause of poisoning; a second possible cause of poisoning, and so on. Further, treatment, future plan, and evaluations can be given in the output. In one particular embodiment, it is contemplated that the output can provide percentage match of the input data points with one or more poisons.

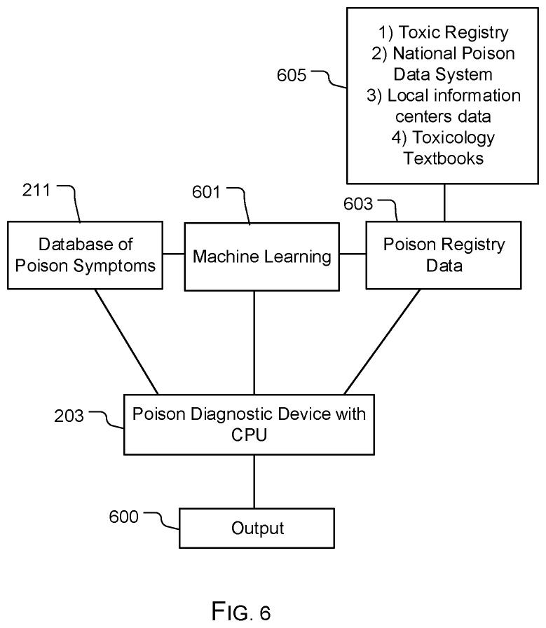

In FIG. 5, a flowchart 501 depicts the method of use of system 201. During use, the data points associated with the plurality of input criteria are put into the display/interface, as shown with box 503. The system, including any necessary software and hardware, is configured to process the data points to correlate to the database and provide an output, as shown with boxes 505, 507. The output provides one or more possible poisons, as shown with box 509.

The system, devices, methods, software, and the like can be used with known poisons and adapted for use with additionally discovered poisons. Further, the system can be updated as needed to maintain keep up with growing science.

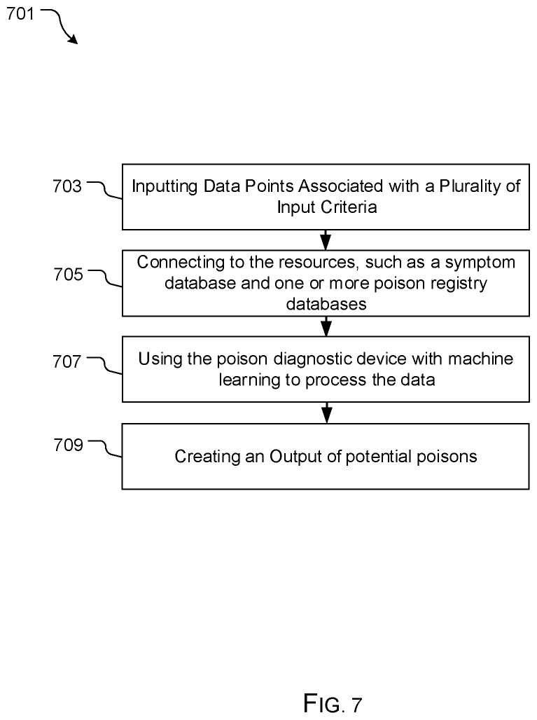

In FIG. 6, a schematic depicts resources contemplated for connection with the poison diagnostic device 203. It should be appreciated that the system can connect to any number of databases and utilize any known or future technology to better create an output 600 for diagnosis of a patient. For example, it is contemplated that the device 203 can communicate with one or more databases of poison symptoms 211 and one or more poison registry data 603. Poison registry data 603 can be from any source, private or governmental, including sources 605 such as: (1) toxic registries such as The ToxIC Qualified Clinical Data Registry (TQCDR); (2) The National Poison Data System (NPDS); (3) Local information centers data; and (4) toxicology textbooks. The system can further utilize machine learning 601 technology to process data with device 203. Machine learning utilizes artificial intelligence to learn systems from data, identify patterns, and make decisions with minimal human intervention. It should be appreciated that the system can match different results from various sources together or report the results from various sources separately. For example, the final suggested diagnosis obtained from textbooks may report separately or may combine with the obtained result of the machine learning process.

In FIG. 7, a flowchart 701 further depicts the method associated with the system of the present invention. As shown, a physician will input data points that are associated with a plurality of criteria into the device, as shown with box 703. The device will connect to any of the plurality of resources shown in FIG. 6 to process the data with machine learning technology, as shown with boxes 705, 707. An output of potential poisons will be provided by the device, as shown with box 709.

Since signs and symptoms or clinical or paraclinical data of poisoning or toxic exposures may have some overlap to each other, we are trying to choose and find the most common early clinical presentation features for every poison or toxins. For this purpose, we will use data of the ToxIC Registry and the National Poison Data System (NPDS) or local information centers data or searching in most popular toxicology textbooks (Gold frank toxicology textbook or critical care in toxicology), or we will explore in searching poisoning journals. Moreover, after getting data from the sources mentioned above especially those derived from For determining which signs and symptoms are most common and have better clinically value, we will use machine learning for analyzing data obtained from the ToxIC Registry and the National Poison Data System (NPDS) or local information centers data. Machine learning has used in other science, but its use in toxicology data is minimal. So, we will use it for analysis of our primary data before selection in the final version of the software. This software will help physician for better toxidrome diagnosis. We will try to integrate this software with the previous one if the outcome is promising.

It is contemplated that some of the poisons included are common substances or pharmaceutical drugs or illicit drugs in the US which cause poisoning for the human. Such as Acetaminophen, Salicylates, NSAIDs (Non steroids anti-inflammatory drugs), Digoxin, Anti-depressants (Tricyclic antidepressant, SSRIs, . . . ), Opioids (Heroin, Methadone, Fentanyl, etc.), Carbon monoxide, anti-hypertensive drugs (Beta blockers, calcium channel blockers, diuretics, etc.,), Pesticides (Organophosphate, Aluminum phosphide, etc,). Antipsychotics (phenothiazines, haloperidol, etc.,), heavy metals poisoning (lead, thallium, Iron, mercury, etc.,), toxic alcohols (methanol, ethylene glycol, etc.,), anti-hyperglycemic agents (insulin, metformin, etc.,), benzodiazepines, etc.

The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. Although the present embodiments are shown above, they are not limited to just these embodiments, but are amenable to various changes and modifications without departing from the spirit thereof.

What is claimed is:

1. A poison diagnostic device, comprising:
a central processing unit in communication with a database and having machine learning technology incorporated therein;
an interface configured to provide a display, the interface configured to communicate with the central processor;
one or more poison registry databases in communication with the central processing unit;
a plurality of data stored within the database and relating to poison symptoms; and
the interface having a plurality of input criteria associated with poison symptoms;
wherein the central processing unit is configured to receive a plurality of data points associated with the plurality of input criteria and correlate the plurality of data points to the database to provide an output; and
wherein the output is a diagnostic sheet providing a correlation between the plurality of data points and possible poison causes.

2. A method of diagnosing poison, the method comprising:
providing a central processing unit connected to a database, the database having a plurality of data associated with poison symptoms;
providing an interface with a display and having a plurality of input criteria associated with poison symptoms;
receiving a plurality of data points associated with the plurality of input criteria; and
correlating the plurality of data points to the database to provide an output through machine learning technology;
wherein the output is a diagnostic sheet providing a correlation between the plurality of data points and possible poison causes.
